(19) United States
(12) Patent Application Publication
(10) Pub. No.: US 2009/0125097 A1
(43) Pub. Date: May 14, 2009

(54) Device and Method for Stent Graft Fenestration in Situ

(75) Inventors: Walter Bruszewski, Guerneville, CA (US); Matthew Rust, North Vancouver (CA); Trevor Greenan, Santa Rosa, CA (US); Masoumeh Mafi, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/939,106

(22) Filed: Nov. 13, 2007

(51) Int. Cl. A61F 2/06; A61M 29/02; A61B 17/34

(52) U.S. Cl. 623/1.23; 606/194; 606/185

(57) ABSTRACT

An anchoring balloon of an anchoring balloon catheter is advanced through the branch vessel to be adjacent to the main stent graft within a main vessel. The anchoring balloon is inflated to center an inner member of the anchoring balloon catheter within the branch vessel and to anchor the anchoring balloon within the branch vessel. A needle assembly is advanced to pierce the graft material of the main stent graft with a needle forming a needle hole in the graft material. A dilator assembly is advanced to dilate the needle hole with a dilator.

### BACKGROUND OF THE INVENTION

1. Field of the Invention

The present invention relates generally to an intra-vascular device and method. More particularly, the present invention relates to a device and method for treatment of intra-vascular diseases.

2. Description of the Related Art

FIG. 1 is a perspective view, partially cutaway, of a needle guiding balloon catheter assembly 100 for fenestrating a main stent graft 102 in accordance with the prior art. Referring now to FIG. 1, an aorta 104 includes an aneurysm 106. Branching off aorta 104 are three branch vessels 108, 110, 112, e.g., the subclavian, the common carotid, and the brachiocephalic trunk.

Main stent graft 102 seals against aorta 104 above and below, e.g., proximally and distally to, aneurysm 106 (only the top of which is shown). Accordingly, fluid flows through the lumen of main stent graft 102 thus bypassing and excluding aneurysm 106.

Due to the variations in the structure and geometry of the anatomy of aorta 104 and branch vessels 108, 110, 112 among a patient population, a fixed design of main stent graft 102 with collateral conduits to provide fluid flow to branch vessels 108, 110, 112 is precluded. As illustrated in FIG. 1, a proposed solution is to fenestrate main stent graft 102 externally by advancing a needle guiding balloon catheter assembly 100 through branch vessel 108 and to main stent graft 102.

Needle guiding balloon catheter assembly 100 includes an elliptical balloon catheter 114 (elliptical in cross section) and a hollow needle 116 affixed to a hollow slideable inner member 117. Elliptical balloon catheter 114 includes an shaft member 118 having an elliptical balloon 120 mounted thereto on a distal end of shaft member 118. Shaft member 118 defines a lumen through which needle 116 is advanced. Needle 116 and inner member 117 are hollow, e.g., tubular, and define a lumen through which a guidewire 122 is passed.

Once located adjacent to main stent graft 102, elliptical balloon 120 is inflated to center shaft member 118 and needle 116. Needle 116 is then advanced to fenestrate (pierce) main stent graft 102 forming a needle hole 124 therein. Guide wire 122 is then passed through the lumen of needle 116 and into main stent graft 102.

Wires, needles, and guiding catheters are then used to dilate needle hole 124 to enable insertion of a dilating balloon. For example, a 0.014″ guide wire is inserted, followed by a 20 ga. needle, followed by a small cutting balloon, followed by a 7 F sheath, followed by two wires to further enlarge the hole, followed by the cutting balloon again, before deploying a 7 mm stent in the fenestration.

However, elliptical balloon 120 is elliptical in cross sectional shape and thus does not ensure that needle 116 is centered or parallel with the axis of branch vessel 108. Further, the elliptical design of elliptical balloon 120 is not consistent with an anchoring function. Accordingly, the puncture force applied to needle 116 in relation to shaft member 118 can be translated into a reaction force which displaces shaft member 118 including elliptical balloon 120. Further, a misapplication of the puncture force to needle 116 can cause needle 116 to deflect from the desired puncture location on main stent graft 102 and into the wall of the ostium of branch vessel 108.

### SUMMARY OF THE INVENTION

In accordance with one example, a method includes deploying a main stent graft within a main vessel covering the ostium to a branch vessel branching from the main vessel, the main stent graft including a graft material. An anchoring balloon of an anchoring balloon catheter is advanced through the branch vessel to be adjacent to the main stent graft, the anchoring balloon catheter further including an inner member defining an inner member lumen therein. The anchoring balloon is inflated to center the inner member approximately parallel with an axis of the branch vessel and to anchor the anchoring balloon within the branch vessel. A needle assembly located within a dilator assembly located within the inner member lumen is advanced to pierce the graft material of the main stent graft with a needle of the needle assembly forming a needle hole in the graft material. The dilator assembly is advanced to dilate the needle hole with a dilator of the dilator assembly.

These and other features according to the present invention will be more readily apparent from the detailed description set forth below taken in conjunction with the accompanying drawings.

### BRIEF DESCRIPTION OF THE DRAWINGS

FIG. 1 is a perspective view, partially cutaway, of a needle guiding balloon catheter assembly for fenestrating a main stent graft in accordance with the prior art;

FIG. 2 is a perspective view of an anchoring balloon catheter in accordance with one embodiment;

FIG. 3 is a cross-sectional view of the anchoring balloon catheter of FIG. 2;

FIG. 4 is a cross-sectional perspective view of a needle dilator guiding catheter assembly in accordance with one embodiment;

FIG. 5 is a perspective view of a needle assembly of the needle dilator guiding catheter assembly of FIG. 4 in accordance with one embodiment;

FIGS. 6 and 7 are cross-sectional views of a slotted hypotube of the needle assembly of FIG. 5 at lines VI-VI and VII-VII, respectively;

FIG. 8 is a perspective view of a dilator assembly of the needle dilator guiding catheter assembly of FIG. 4 in accordance with one embodiment;

FIG. 9 is a perspective view of a dilator of the dilator assembly of FIG. 8 in accordance with one embodiment;

FIG. 10 is a side view of the dilator of FIG. 9;

FIG. 11 is a distal end view of the dilator of FIG. 10;

FIG. 12 is a cross-sectional view of the dilator of FIG. 11 along the line XII-XII of FIG. 11;

FIG. 13 is an enlarged side view of a region XIII of the dilator of FIG. 10;

FIG. 14 is a perspective view of a hybrid needle-dilator in accordance with one embodiment;

FIG. 15 is a side view of the hybrid needle-dilator of FIG. 14;

FIG. 16 is an enlarged perspective view of the region XVI of the hybrid needle-dilator of FIG. 14;

FIG. 17 is a perspective view of a hybrid needle-dilator in accordance with one embodiment;

FIG. 18 is a side view of the hybrid needle-dilator of FIG. 17;

FIG. 19 is a distal end view of the hybrid needle-dilator of FIG. 18;

FIG. 20 is a cross-sectional view of the hybrid needle-dilator of FIG. 19 at line XX-XX;

FIG. 21 is an enlarged side view of a region XXI of the hybrid needle-dilator of FIG. 18;

FIG. 22 is a perspective view of a T-coupler assembly for a user interface for manipulating the needle dilator guiding catheter assembly of FIG. 4 in accordance with one embodiment;

FIGS. 23 and 24 are perspective views, partially cutaway, of an actuator handle of a user interface;

FIGS. 25 and 26 are perspective views of the user interface in accordance with one embodiment; and

FIGS. 27, 28, 29, 30, 31, 32, 33, and 34 are perspective schematic views illustrating formation of a collateral opening in a main stent graft and deployment of a branch prosthesis within the collateral opening in accordance with one embodiment.

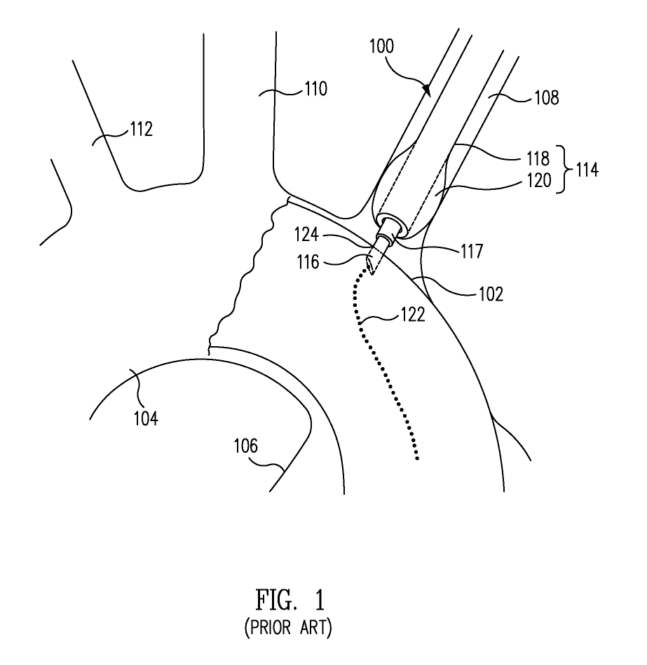

In the following description, the same or similar elements are labeled with the same or similar reference numbers.

### DETAILED DESCRIPTION

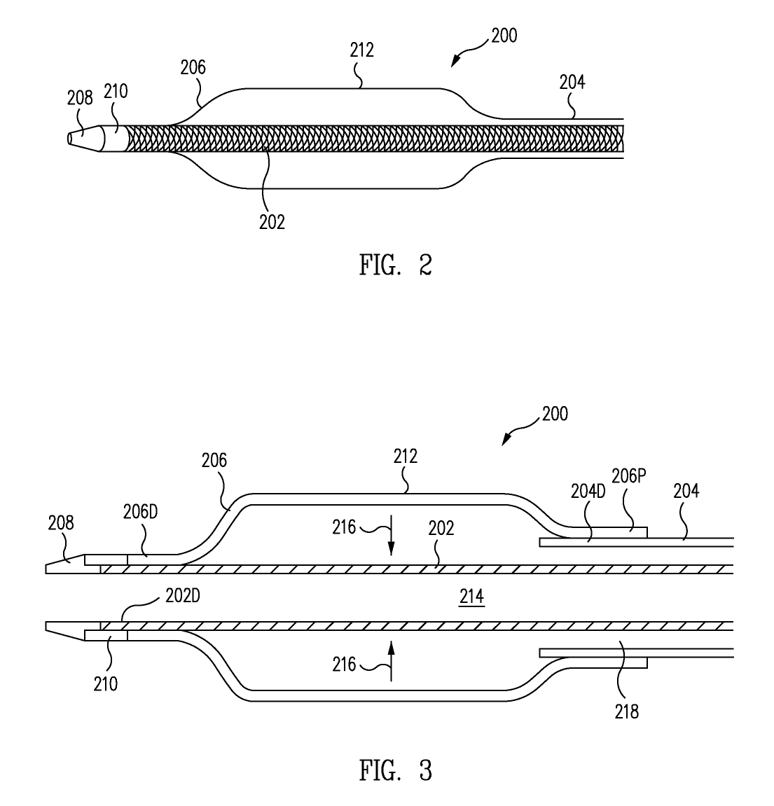

FIG. 2 is a perspective view of an anchoring balloon catheter 200 in accordance with one embodiment. FIG. 3 is a cross-sectional view of anchoring balloon catheter 200 of FIG. 2. Referring now to FIGS. 2 and 3 together, anchoring balloon catheter 200 includes an inner member 202, an outer member 204, an anchoring balloon 206, a soft tapered tip 208 and a marker band 210.

Anchoring balloon 206 is a compliant balloon, i.e., has a low modulus of elasticity. In the view of FIGS. 2 and 3, anchoring balloon 206 is illustrated as inflated. As illustrated, anchoring balloon 206 is cylindrical, sometimes called rectangular. More particularly, anchoring balloon 206 has an approximately cylindrical outer surface 212.

By forming anchoring balloon 206 as a compliant cylindrical balloon, anchoring balloon 206 anchors anchoring balloon catheter 200 within and aligned with the axis of the branch vessel as discussed further below.

The distal end of 206D of anchoring balloon 206 is mounted on inner member 202. Inner member 202 is a hollow tubular member and defines an inner member lumen 214 therein. To prevent collapse of inner member 202 and constriction of inner member lumen 214 when anchoring balloon 206 is inflated, inner member 202 is formed of a thermoplastic elastomer impregnated stainless steel braided material, e.g., ultra thin wall stainless steel ribbon braid. More particularly, when anchoring balloon 206 is inflated, among other forces an inward radial force is exerted on inner member 202 as indicated by the arrows 216. Inner member 202 has sufficient strength to prevent collapse from the inward radial force allowing a needle/dilator assembly to be passed through and moved within inner member lumen 214 as discussed further below. Illustratively, inner member 202 has sufficient strength to withstand a balloon pressure of 2 atm.

Distal end 206D of anchoring balloon 206 is mounted and sealed to a distal end 202D of inner member 202. A proximal end 206P of anchoring balloon 206 is mounted and sealed to a distal end 204D of outer member 204.

As used herein, the proximal end of a prosthesis such as a stent-graft is the end closest to the heart via the path of blood flow whereas the distal end is the end furthest away from the heart during deployment. In contrast and of note, the distal end of the catheter is usually identified to the end that is farthest from the operator (handle) while the proximal end of the catheter is the end nearest the operator (handle). For purposes of clarity of discussion, as used herein, the distal end of the catheter is the end that is farthest from the operator (the end furthest from the handle) while the distal end of the prosthesis is the end nearest the operator (the end nearest the handle), i.e., the distal end of the catheter and the proximal end of the stent-graft are the ends furthest from the handle while the proximal end of the catheter and the distal end of the stent-graft are the ends nearest the handle. However, those of skill in the art will understand that depending upon the access location, the stent-graft and delivery system description may be consistent or opposite in actual usage.

A balloon inflation lumen 218 is defined within the annular space between inner member 202 and outer member 204. Balloon inflation lumen 218 is in fluid communication with anchoring balloon 206 facilitating inflation and deflation thereof.

Soft tapered tip 208 is mounted to distal end 202D of inner member 202 facilitating advancement of anchoring balloon catheter 200 through the branch vessel as discussed further below. Marker band 210, e.g., a radiopaque material, facilitates positioning of anchoring balloon catheter 200.

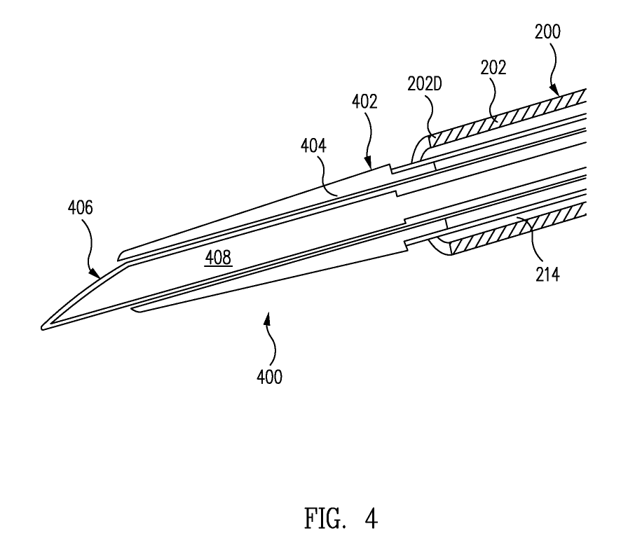

FIG. 4 is a perspective cross-sectional view of a needle dilator guiding catheter assembly 400 in accordance with one embodiment. Referring now to FIG. 4, needle dilator guiding catheter assembly 400 includes anchoring balloon catheter 200 as discussed above in reference to FIGS. 2 and 3. In the view of FIG. 4, distal end 202D of inner member 202 is illustrated and tapered tip 208 is not illustrated for purposes of clarity. As discussed above, inner member 202 defines inner member lumen 214 therein.

Located within inner member lumen 214 and protruding distally from inner member 202 is a dilator assembly 402. Dilator assembly 402 is a hollow tubular member and defines a dilator assembly lumen 404 therein.

Located within dilator assembly lumen 404 and protruding distally from dilator assembly 402 is a needle assembly 406. Needle assembly 406 is a hollow tubular member and defines a needle assembly lumen 408 therein, sometimes called a guide wire lumen.

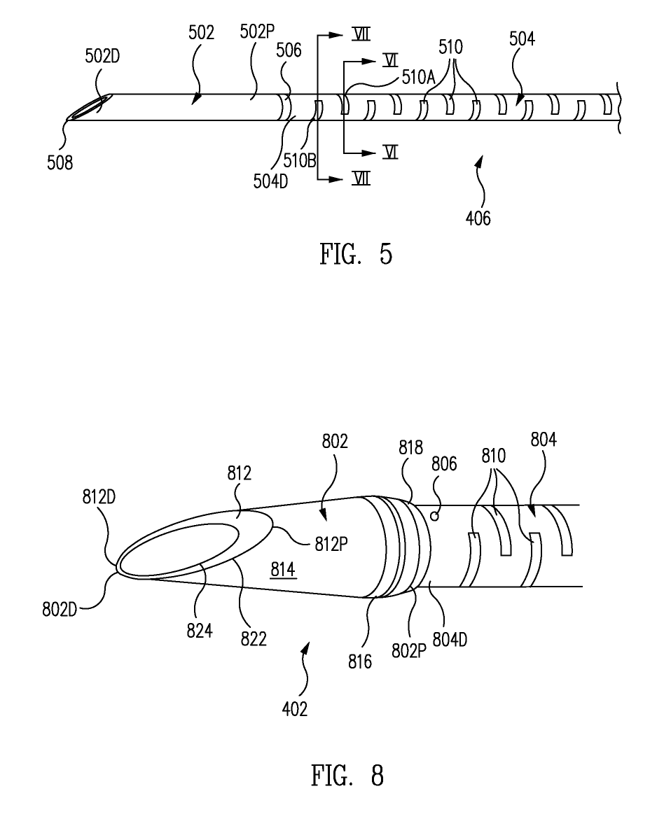

FIG. 5 is a perspective view of needle assembly 406 of needle dilator guiding catheter assembly 400 of FIG. 4 in accordance with one embodiment. Referring now to FIG. 5, needle assembly 406 includes a hollow needle 502, similar to a conventional hypodermic needle, and a slotted hypotube 504. More particularly, a proximal end 502P of needle 502 is mounted to a distal end 504D of slotted hypotube 504 at a laser weld 506, although adhesive or another fastener can be used in another example. Needle 502 includes a sharp tip 508 at a distal end 502D thereof. Sharp tip 508 is similar or identical to the sharp tip of a conventional hypodermic needle as is well known in the art and so is not discussed in detail. Needle 502 is used to form the initial puncture (needle hole) in the graft material of the main stent graft as discussed further below.

Slotted hypotube 504 is a polyethylene terephthalate (PET) jacketed (shrunken onto the hypotube or co-extruded) hypotube, i.e., a hollow tube, and includes a plurality of slots 510, sometimes called alternated laser C-slots. Polymers other than PET can be used such as nylon, Ultem, PolyTetraFluoroEthylene (PTFE) or other polymer that functions to prevent loss of column integrity with high axial loading when slotted hypotube 504 is pushed around a curve.

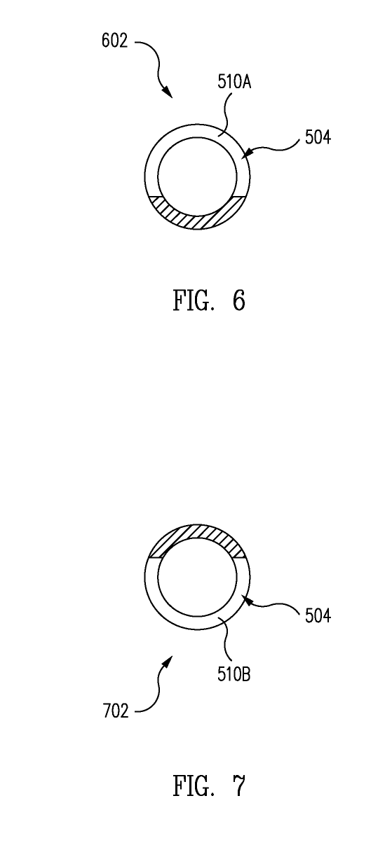

FIGS. 6 and 7 are cross-sectional views of slotted hypotube 504 of needle assembly 406 of FIG. 5 along the lines VI-VI and VII-VII, respectively. In the view of FIG. 6 and 7, slotted hypotube 504 is illustrated as a single layer for simplicity of presentation although it is to be understood that slotted hypotube 504 includes a central hypotube coated on the outside (sometimes called jacketed) with PET.

Referring now to FIGS. 5, 6, and 7 together, slots 510 include a first slot 510A and a second slot 510B. First slot 510A is alternated with a second slot 510B. More particularly, first slot 510A is laser cut into slotted hypotube 504 from a first direction 602, e.g., downwards and second slot 510B is laser cut into slotted hypotube 504 from a second direction 702, e.g., upwards, opposite first direction 602. Accordingly, slots 510A, 510B (and slots 510 generally) are alternated 180 degrees from one another.

By forming slotted hypotube 504 with alternated laser C-slots 510, slotted hypotube 504 has the combined properties of strength in compression to advance needle 502, strength in tension to retract needle 502, and flexibility to negotiate turns, e.g., of 1 cm radius.

FIG. 8 is a perspective view of dilator assembly 402 of needle dilator guiding catheter assembly 400 of FIG. 4 in accordance with one embodiment. Referring now to FIG. 8, dilator assembly 402 includes a dilator 802 and a slotted hypotube 804. More particularly, a proximal end 802P of dilator 802 is mounted to a distal end 804D of slotted hypotube 804 at a plurality of laser spot welds 806, e.g., three spot welds offset from one another by 120°, although adhesive or another fastener is used in another example.

Slotted hypotube 804 is substantially similar to slotted hypotube 504 of FIGS. 5, 6 and 7 except has a larger diameter to accommodate needle assembly 406 therein. More particularly, slotted hypotube 804 is a PET jacketed hypotube and includes a plurality of slots 810, sometimes called alternated laser C-slots. Slots 810 are similar to slots 510 as discussed above, the discussion of which is herein incorporated by reference.

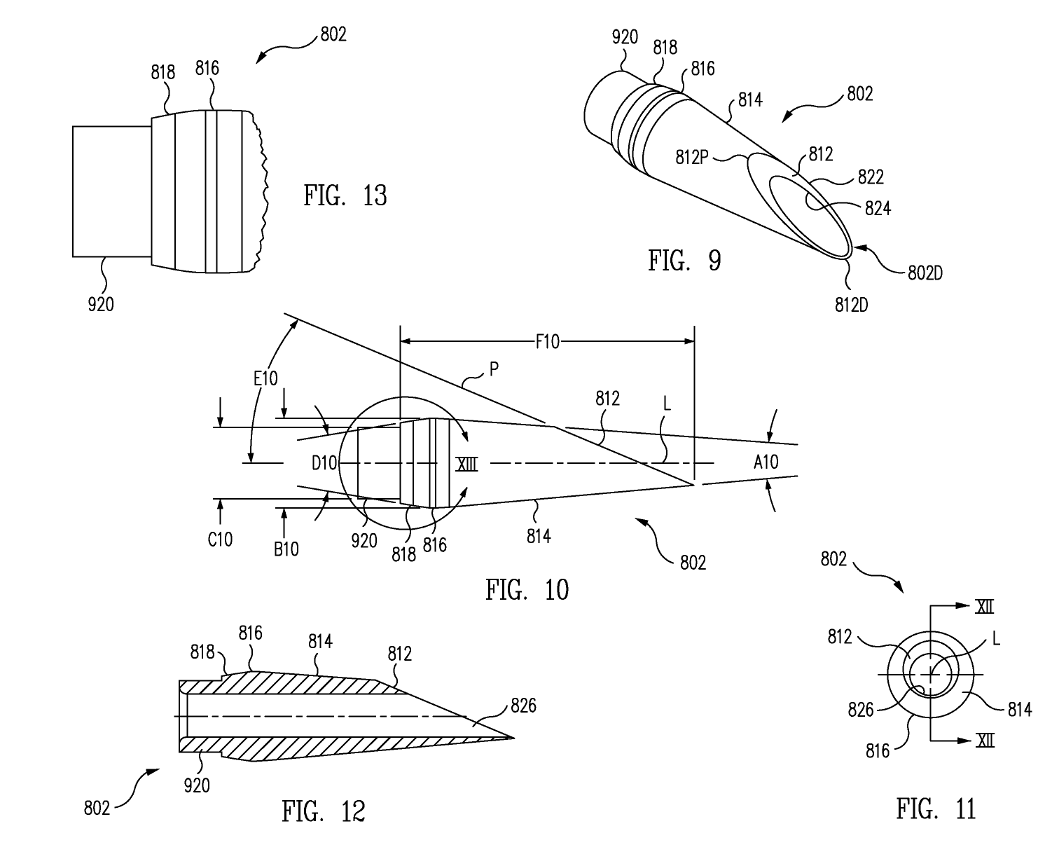

Dilator 802 is used to dilate (enlarge) the needle hole formed by needle 502 as discussed further below. In one example, dilator 802 is formed of metal. Dilator 802 includes a bevel 812, a distal tapering exterior surface 814, a shoulder 816, a proximal tapering exterior surface 818 and a hypotube collar. The hypotube collar fits inside of slotted hypotube 804 and so is not illustrated in the view of FIG. 8, but is illustrated as hypotube collar 920 in FIGS. 9,10, 12 and 13.

FIG. 9 is a perspective view of dilator 802 of dilator assembly 402 of FIG. 8 in accordance with one embodiment. FIG. 10 is a side view of dilator 802 of FIG. 9. FIG. 11 is a distal end view of dilator 802 of FIG. 10. FIG. 12 is a cross-sectional view of dilator 802 of FIG. 11 along the line XII-XII. FIG. 13 is an enlarged side view of a region XII of dilator 802 of FIG. 10.

Referring now to FIGS. 8, 9, 10,11, 12, and 13 together, bevel 812 is an angled flat cut in dilator 802. More particularly, dilator 802 includes a longitudinal axis L. Bevel 812 is a flat cut, i.e., lies in a plane P (projecting perpendicularly out of the page of FIG. 10). Further, bevel 812 is at an angle E10 with respect to longitudinal axis L.

Bevel 812 is the surface created by the intersection of plane P and a right circular cone having a cylindrical lumen therein, plane P being angled at an angle of greater than 0 degrees and less than 90 degrees relative to longitudinal axis L.

More particularly, bevel 812 is an elliptical annular surface having a varied thickness. An outer periphery 822 of bevel 812 is the ellipse created by the intersection of plane P and the outer surface of a right circular cone, which is defined by distal tapering exterior surface 814 in this example. An inner periphery 824 of bevel 812 is the ellipse created by the intersection of the same plane P and an inner cylindrical surface 826 that defines dilator assembly lumen 404.

The distance between outer periphery 822 and inner periphery 824 of bevel 812 is minimum at a distal end 812D of bevel 812 and maximum at a proximal end 812P of bevel 812 and gradually increases between distal end 812D and proximal end 812P.

Bevel 812 defines the distal surface of dilator 802 at distal end 802D of dilator 802. Bevel 812 facilitates insertion of dilator 802 into the needle hole in the graft material as discussed further below.

Distal tapering exterior surface 814 increasingly tapers proximally from bevel 812. More particularly, the diameter of distal tapering exterior surface 814 in a direction perpendicular to longitudinal axis L increases proximally from bevel 812 to have a maximum diameter at shoulder 816. Distal tapering exterior surface 814 extends from bevel 812 to shoulder 816.

Shoulder 816 is the maximum diameter portion of dilator 802. Shoulder 816 is defined at the intersection of distal tapering exterior surface 814 and proximal tapering exterior surface 818. Shoulder 816 is a cylindrical surface that snuggly fits within inner member lumen 214 of inner member 202 of anchoring balloon catheter 200 (see FIG. 4 for example). Accordingly, shoulder 816 functions as a guide insuring that dilator 802 remains aligned with the longitudinal axis of anchoring balloon catheter 200 during advancement and dilation of the graft material as discussed further below.

Further, shoulder 816 has an outer diameter greater than the outer diameter of slotted hypotube 804. Illustratively, shoulder 816 has an outer diameter of 0.1050 inches and the outer diameter of slotted hypotube 804 is 0.095 inches although shoulder 816 and/or slotted hypotube 804 have other outer diameters in other examples. This radial separation between most of the outside diameter of hypotube 804 and inner member 202 minimizes friction between slotted hypotube 804 and inner member 202 thus minimizing the delivery force necessary to advance dilator 802.

Proximal tapering exterior surface 818 is tapered (provides a gradual diameter transition) to provide a smooth transition between slotted hypotube 804 and dilator 802 preventing catching of dilator 802 on the graft material during retraction of dilator 802 as discussed further below.

Proximal tapering exterior surface 818 decreasingly tapers proximally from shoulder 816 and extends from shoulder 816. More particularly, the diameter of proximal tapering exterior surface 818 in a direction perpendicular to longitudinal axis L decreases proximally from shoulder 816 to have a minimum diameter at hypotube collar 920.

The exterior surface of dilator 802, i.e., distal tapering exterior surface 814, shoulder 816, and proximal tapering exterior surface 818, is polished, e.g., having an average roughness (Ra) less than or equal to 8 μin, thus facilitating sliding (minimizing friction between) dilator 802 and the graft material being dilated as discussed further below.

Illustrative specification for the various characteristics illustrated in FIG. 10 are set forth below in Table 1.

TABLE 1

| CHARACTERISTIC | SPECIFICATION | UNIT |
|---|---|---|
| A10 | 9.3 | degrees |
| B10 | 0.1050 | inches |
| C10 | 0.0850 | inches |
| D10 | 22.6 | degrees |
| E10 | 23.0 | degrees |
| F10 | 0.3500 | inches |

In another example, instead of providing a separate needle and dilator, the functionality of a needle and dilator is combined into a hybrid needle-dilator. The hybrid needle-dilator functions to both form the needle hole in the graft material and then dilate the needle hole. The hybrid needle-dilator is mounted to a single slotted hypotube that extends through the inner member lumen of the inner member.

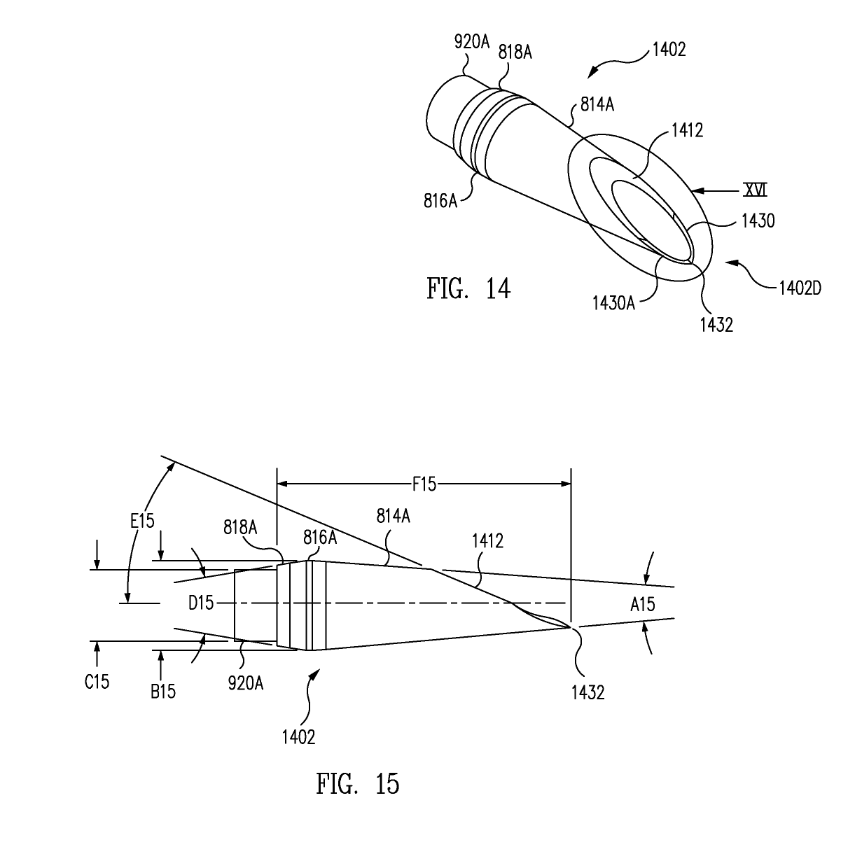

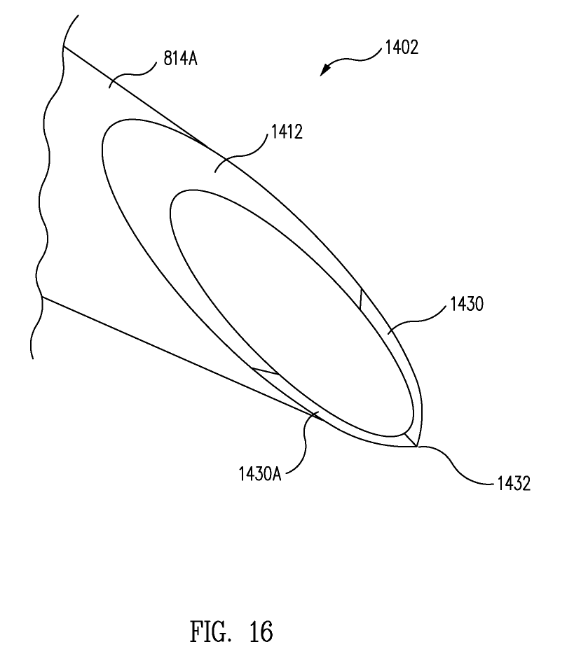

FIG. 14 is a perspective view of hybrid needle-dilator 1402 in accordance with one embodiment. FIG. 15 is a side view of hybrid needle-dilator 1402 of FIG. 14. FIG. 16 is an enlarged perspective view of the region XVI of hybrid needle-dilator 1402 of FIG. 14.

Referring now to FIGS. 14, 15 and 16 together, hybrid needle-dilator 1402, sometimes called a dilator, includes a distal tapering exterior surface 814A, a shoulder 816A, a proximal tapering exterior surface 818A, and a hypotube collar 920A similar to distal tapering exterior surface 814, shoulder 816, proximal tapering exterior surface 818, and hypotube collar 920 of dilator 802 of FIGS. 8, 9, 10, 11, 12, and 13.

However, in accordance with this example, two bevels 1430, 1430A are formed in a bevel 1412 to define a sharp tip 1432 at a distal end 1402D of hybrid needle-dilator 1402. Bevels 1412, 1430, 1430A and sharp tip 1432 are similar to the bevels and sharp tip of a conventional hypodermic needle. Bevels 1430 and 1430A are planar surfaces. These planes are described by two angles: the angle of the plane relative to the plane of the view of FIG. 15 (e.g., 20 degrees); the angle of the plane relative to the longitudinal axis of hybrid needle-dilator 1402 (e.g., 24 degrees).

Illustrative specification for the various characteristics illustrated in FIG. 15 are set forth below in Table 2.

TABLE 2

| CHARACTERISTIC | SPECIFICATION | UNIT |
|---|---|---|
| A15 | 10.0 | degrees |
| B15 | 0.1050 | inches |
| C15 | 0.0850 | inches |
| D15 | 22.6 | degrees |
| E15 | 18.0 | degrees |
| F15 | 0.3682 | inches |

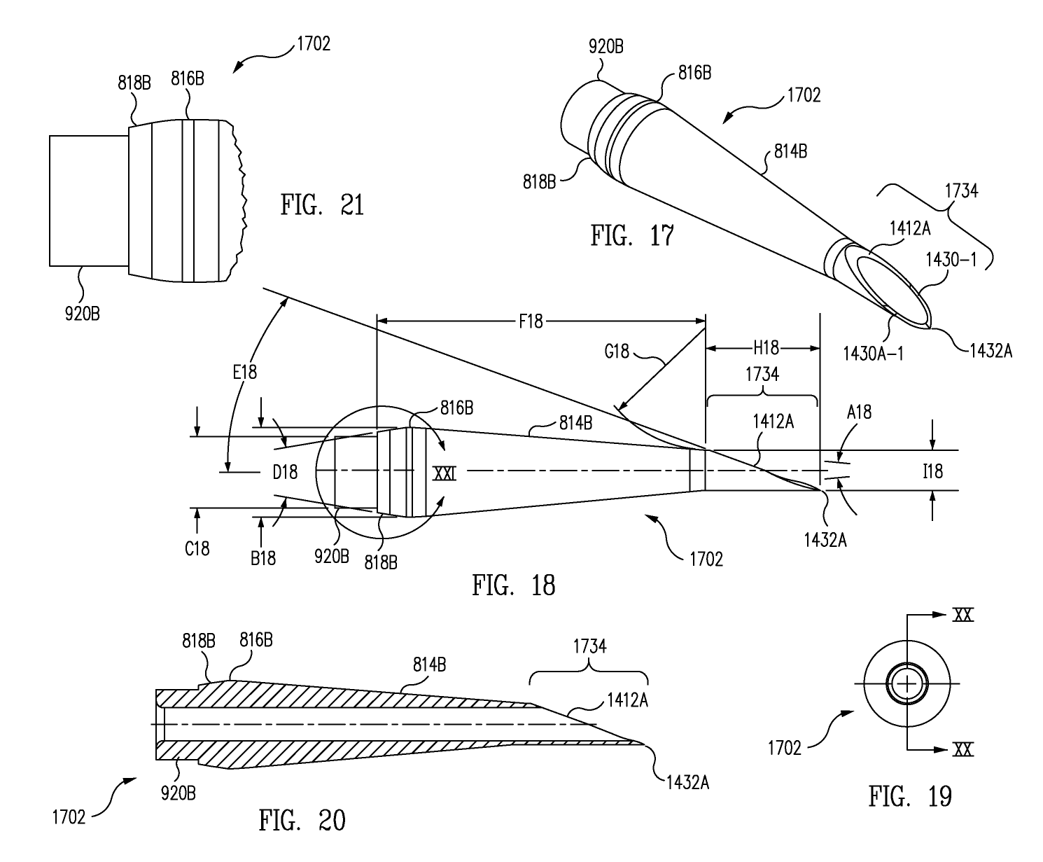

FIG. 17 is a perspective view of hybrid needle-dilator 1702 in accordance with one embodiment. FIG. 18 is a side view of hybrid needle-dilator 1702 of FIG. 17. FIG. 19 is a distal end view of hybrid needle-dilator 1702 of FIG. 18. FIG. 20 is a cross-sectional view of hybrid needle-dilator 1702 of FIG. 19 along the line XX-XX. FIG. 21 is an enlarged side view of a region XXI of hybrid needle-dilator 1702 of FIG. 18.

Referring now to FIGS. 17, 18, 19, 20, and 21 together, hybrid needle-dilator 1702 includes a bevel 1412A, a distal tapering exterior surface 814B, a shoulder 816B, a proximal tapering exterior surface 818B, and a hypotube collar 920B similar to bevel 1412, distal tapering exterior surface 814A, shoulder 816A, proximal tapering exterior surface 818A, and hypotube collar 920A of hybrid needle-dilator 1402 of FIGS. 14, 15, 16.

However, in accordance with this example, distal tapering exterior surface 814B extends between a distal needle portion 1734 of hybrid needle-dilator 1702 and shoulder 816B. Distal needle portion 1734 has a uniform diameter similar to a conventional hypodermic needle. Further, distal needle portion 1734 includes bevel 1412A, and bevels 1430-1, 1430A-1 that define a sharp tip 1432A. Bevels 1430-1, 1430A-1 and sharp tip 1432A are similar to the bevels and sharp tip of a conventional hypodermic needle. Bevels 1430-1 and 1430A-1 are planar surfaces. These planes are described by two angles: the angle of the plane relative to the plane of the view of FIG. 18 (e.g., 30 degrees); the angle of the plane relative to the longitudinal axis of hybrid needle-dilator 1702 (e.g., 28 degrees).

Illustrative specification for the various characteristics illustrated in FIG. 18 are set forth below in Table 3.

TABLE 3

| CHARACTERISTIC | SPECIFICATION | UNIT |
|---|---|---|
| A18 | 10.0 | degrees |
| B18 | 0.1050 | inches |
| C18 | 0.0850 | inches |
| D18 | 22.6 | degrees |
| E18 | 18.0 | degrees |
| F18 | 0.3838 | inches |
| G18 | R.2000 | inches |
| H18 | 0.1350 | inches |
| I18 | .0480 | inches |

Although various specifications are set forth in tables 1, 2, and 3, other specifications, e.g., angles and/or lengths, are possible, as long as safety or functionality is not impaired. For example, longer dilator length may result in lower dilation force, however, the dilator may not function safely in a small (22-24 mm) stent graft due to increased risk of inadvertent contact. Conversely, shorter dilator length may be safer due to decreased risk of inadvertent contact, however, may result in greater dilation force that can de-stabilize the column of the slotted hypotube on which the dilator is mounted.

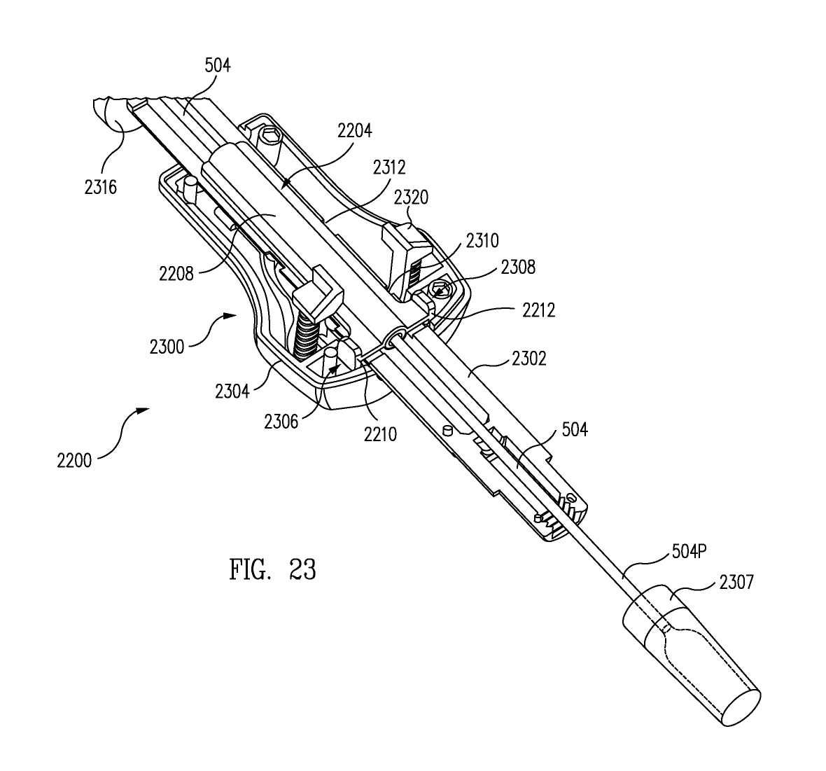

Referring back to FIG. 4, in accordance with one example, a user interface for manipulating needle dilator guiding catheter assembly 400 is presented as set forth below in reference to FIGS. 23, 24, 25 and 26.

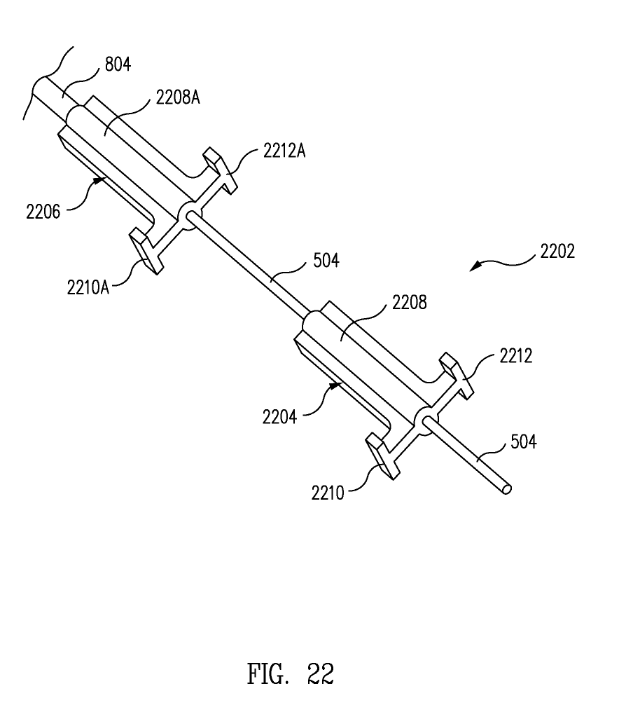

FIG. 22 is a perspective view of a T-coupler assembly 2202 for a user interface for manipulating needle dilator guiding catheter assembly 400 of FIG. 4 in accordance with one embodiment. Referring now to FIG. 22, T-coupler assembly 2202 includes a needle T-coupler 2204 and a dilator T-coupler 2206. Needle T-coupler 2204 is substantially similar or identical to dilator T-coupler 2206.

Referring now to FIGS. 4 and 22 together, needle T-coupler 2204 includes a T-coupler body 2208 and T-coupler ears 2210, 2212 protruding radially from T-coupler body 2208. T-coupler body 2208 has a cylinder opening extending longitudinally therethrough similar to a tube. Slotted hypotube 504 extends through and is mounted within the cylindrical opening in T-coupler body 2208, e.g., with adhesive. Accordingly, longitudinal motion of needle T-coupler 2204 translates to longitudinal motion of slotted hypotube 504 and needle assembly 406.

Similarly, dilator T-coupler 2206 includes a T-coupler body 2208A and T-coupler ears 2210A, 2212A protruding radially from T-coupler body 2208A. T-coupler body 2208A has a cylinder opening extending longitudinally therethrough similar to a tube. The proximal end of slotted hypotube 804 extends into and is mounted within the cylindrical opening in T-coupler body 2208A, e.g., with adhesive. Accordingly, longitudinal motion of dilator T-coupler 2206 translates to longitudinal motion of slotted hypotube 804 and dilator assembly 402.

Slotted hypotube 504 extends distally from needle T-coupler 2204 and into the proximal end of slotted hypotube 804 and through dilator T-coupler 2206.

Slotted hypotubes 504, 804 are illustrated as having an absence of slots in the view of FIG. 22. Generally, slotted hypotubes 504, 804 are formed with slots on the distal portion of slotted hypotubes 504, 804 that need to bend during advancement of slotted hypotubes 504, 804. As it is unnecessary for slotted hypotubes 504, 804 to bend within the user interface, the proximal portion of slotted hypotubes 504, 804 have an absence of slots, i.e., are solid tubular members.

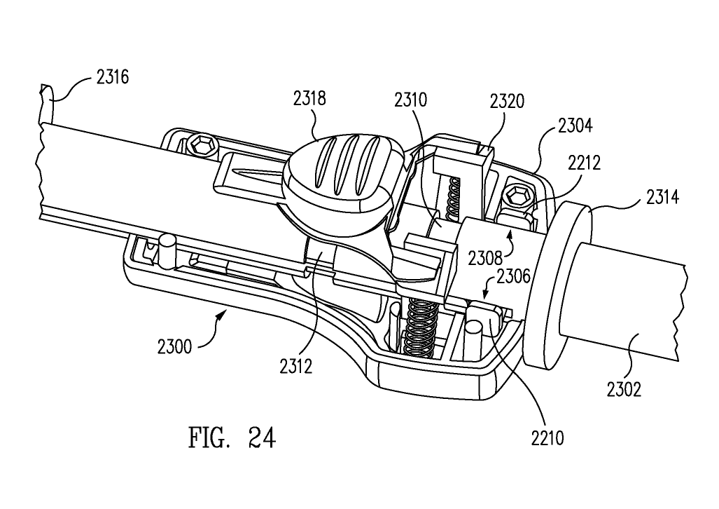

FIGS. 23 and 24 are perspective views, partially cutaway, of a needle actuator handle 2300 of a user interface 2200. Referring now to FIGS. 4, 5, 23 and 24 together, needle actuator handle 2300 is used to longitudinally translate, sometimes called move or slide, needle T-coupler 2204 and thus slotted hypotube 504 and needle 502 with respect to anchoring balloon catheter 200 and each other.

More particularly, T-coupler ears 2210, 2212 extend from T-coupler body 2208 and through a slot within an actuator shaft 2302 of user interface 2200. Actuator shaft 2302, which is fixed to anchoring balloon catheter 200 as discussed below, is illustrated in cross-section in the view of FIG. 23. Needle actuator handle 2300 further includes a handle housing 2304 having pockets 2306, 2308 in which T-coupler ears 2210, 2212 are located and secured. In this manner, needle assembly 406 is coupled to needle actuator handle 2300. A luer adaptor 2307 is mounted to a proximal end 504P of slotted hypotube 504. Luer adaptor 2307 facilitates entry of the guidewire into the lumen of slotted hypotube 504.

Actuator shaft 2302 includes a retracted position locking engagement groove 2310 and an extended position locking engagement groove 2312. Actuator shaft 2302 further includes a proximal stop 2314 and a distal stop 2316, sometimes called needle travel limiters. Proximal stop 2314 is not illustrated in FIG. 23 to allow visualization of features of needle actuator handle 2300.

Proximal stop 2314 sets the proximal limit of motion (travel) of needle actuator handle 2300, i.e., needle actuator handle 2300 can only be moved proximally until needle actuator handle 2300 contacts proximal stop 2314. Similarly, distal stop 2316 sets the distal limit of motion (travel) of needle actuator handle 2300, i.e., needle actuator handle 2300 can only be move distally until needle actuator handle 2300 contacts distal stop 2316. By limiting the maximum distal position of needle actuator handle 2300 and thus the maximum distal advancement of needle 502, over advancement of needle 502 and thus inadvertently puncturing of the opposite side of the graft material and possibly the main vessel, e.g., the aorta, is prevented.

Needle actuator handle 2300 further includes one or more actuation buttons 2318, e.g., two actuation buttons opposite one another. For simplicity of discussion, needle actuator handle 2300 will be discussed herein as having only a single actuation button 2318, although it is to be understood that needle actuator handle 2300 can include two or more actuation buttons.

Actuation button 2318 is engaged with a spring-loaded locking member 2320. Spring-loaded locking member 2320 fits within locking engagement groove 2310, 2312 thus locking needle actuator handle 2300 in the retracted or extended position, respectively.

More particularly, in the views of FIGS. 23, 24, spring-loaded locking member 2320 is located within retracted position locking engagement groove 2310. Accordingly, needle actuator handle 2300 is locked to actuator shaft 2302 thus locking needle 502 in the retracted position. In this manner, inadvertently advancement of needle 502 and the associated inadvertently piercing of the branch vessel during advancement of needle dilator guiding catheter assembly 400 to the treatment site is prevented.

To disengage spring-loaded locking member 2320 from retracted position locking engagement groove 2310, actuation button 2318 is depressed thus moving spring-loaded locking member 2320 out of locking engagement groove 2310. This allows needle actuator handle 2300 to be slid distally on actuator shaft 2302 until needle actuator handle 2300 contacts distal stop 2316. Upon contact of distal stop 2316, spring-loaded locking member 2320 is aligned with extended position locking engagement groove 2312 and is spring-loaded therein.

Similarly, to disengage spring-loaded locking member 2320 from extended position locking engagement groove 2312, actuation button 2318 is depressed thus moving spring-loaded locking member 2320 out of extended position locking engagement groove 2312. This allows needle actuator handle 2300 to be slid proximally on actuator shaft 2302 until needle actuator handle 2300 contacts proximal stop 2314. Upon contact of proximal stop 2314, spring-loaded locking member 2320 is aligned with retracted position locking engagement groove 2310 and is spring-loaded therein.

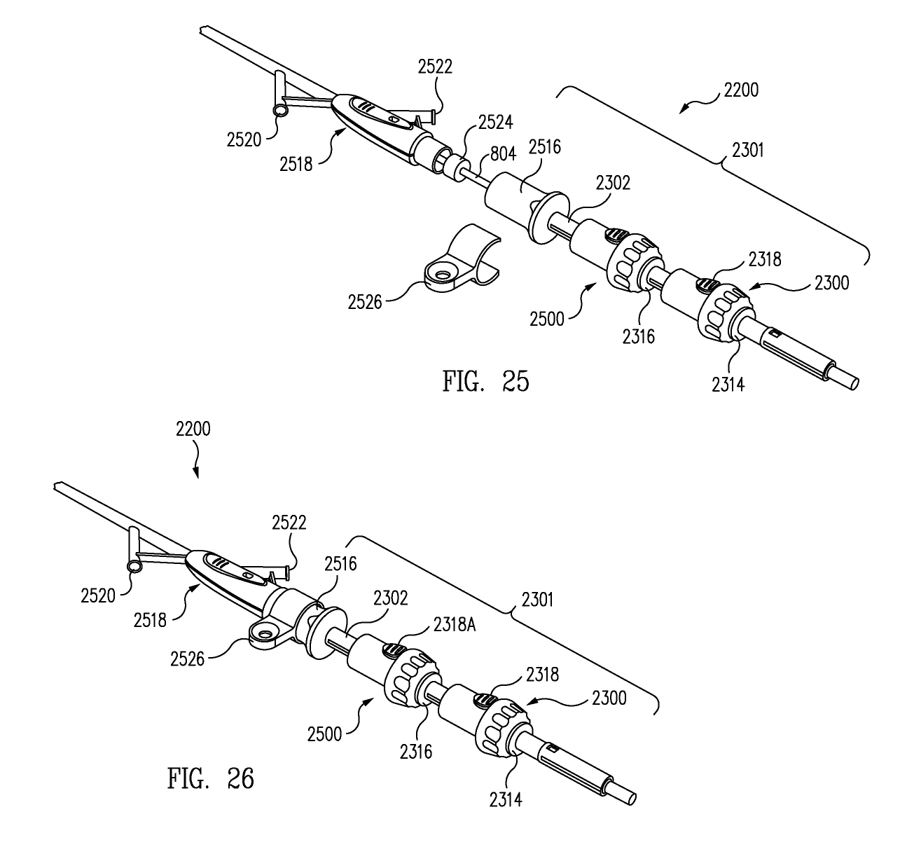

FIGS. 25 and 26 are perspective views of user interface 2200 in accordance with one embodiment. Referring now to FIGS. 3, 4, 5, 8, 22, 25 and 26 together, user interface 2200 further includes a dilator actuator handle 2500 coupled to dilator T-coupler 2206. Dilator actuator handle 2500 is substantially similar or identical to needle actuator handle 2300.

Actuator shaft 2302 further includes a second retracted position locking engagement groove and a second extended position locking engagement groove similar to locking engagement grooves 2310, 2312 for locking dilator actuator handle 2500 in the retracted and extended positions. Further, dilator actuator handle 2500 is coupled to dilator T-coupler 2206 in a substantially similar or identical manner to the coupling of needle actuator handle 2300 to needle T-coupler 2204 and so is not discussed in detail.

One notable difference is that distal stop 2316 forms the proximal stop for dilator actuator handle 2500 and an engagement cap 2516 forms the distal stop for dilator actuator handle 2500. Distal stop 2316 and engagement cap 2516 are sometimes called dilator travel limiters.

Illustratively, actuator shaft 2302, engagement cap 2516, needle actuator handle 2300, dilator actuator handle 2500, needle 502, slotted hypotube 504, dilator 802, and slotted hypotube 804 collectively form a needle dilator assembly 2301.

User interface 2200 further includes an anchor balloon catheter handle 2518. Referring now to FIGS. 2, 3, 25, and 26 together, anchor balloon catheter handle 2518 includes a balloon inflation port 2520 and an inner member lumen flush port 2522.

Balloon inflation port 2520 is in fluid communication with balloon inflation lumen 218 and thus anchoring balloon 206. Accordingly, anchoring balloon 206 is inflated and deflated through balloon inflation port 2520.

Inner member lumen flush port 2522 is in fluid communication with inner member lumen 214 of inner member 202. Accordingly, inner member lumen 214 is flushed through inner member lumen flush port 2522.

A proximal end of inner member 202 extends within and is coupled to anchor balloon catheter handle 2518. Co-axial slotted hypotubes 804, 504 (hypotube 504 concentrically inside of hypotube 804) extend through a fitting 2524 and into anchor balloon catheter handle 2518 and thus into inner member lumen 214 of inner member 202.

Engagement cap 2516 and thus needle dilator assembly 2301 is mounted to anchor balloon catheter handle 2518 through an interference fit, sometimes called a friction fit. Further, a locking snap 2526 locks engagement cap 2516 to anchor balloon catheter handle 2518. In one example, locking snap 2526 includes one or more protruding tabs that pass-through aligned apertures in engagement cap 2516 and anchor balloon catheter handle 2518 locking engagement cap 2516 and needle dilator assembly 2301 to anchor balloon catheter handle 2518.

In this manner, needle dilator assembly 2301 can be removed from anchor balloon catheter 200 and other devices such as a dilating balloon, a branch prosthesis, and branch prosthesis flaring balloon can be inserted into anchor balloon catheter 200 as discussed further below.

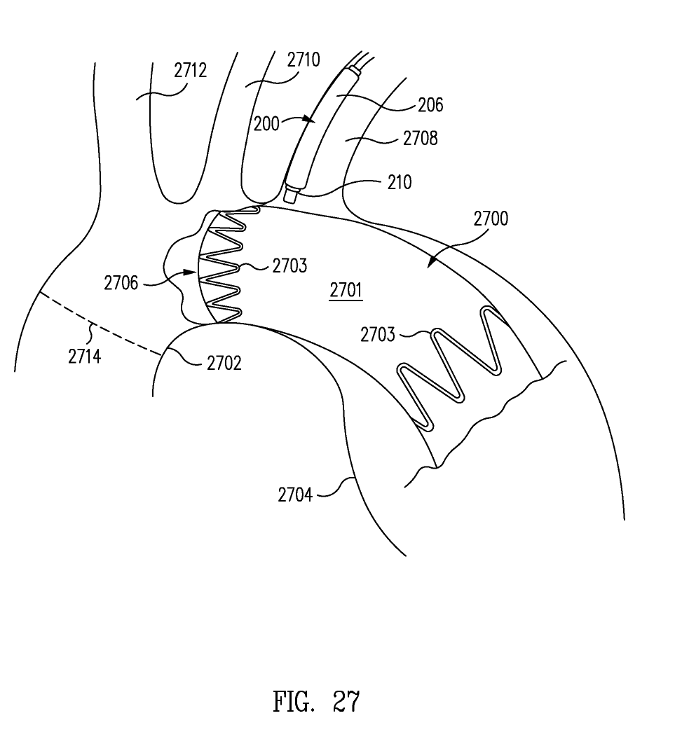

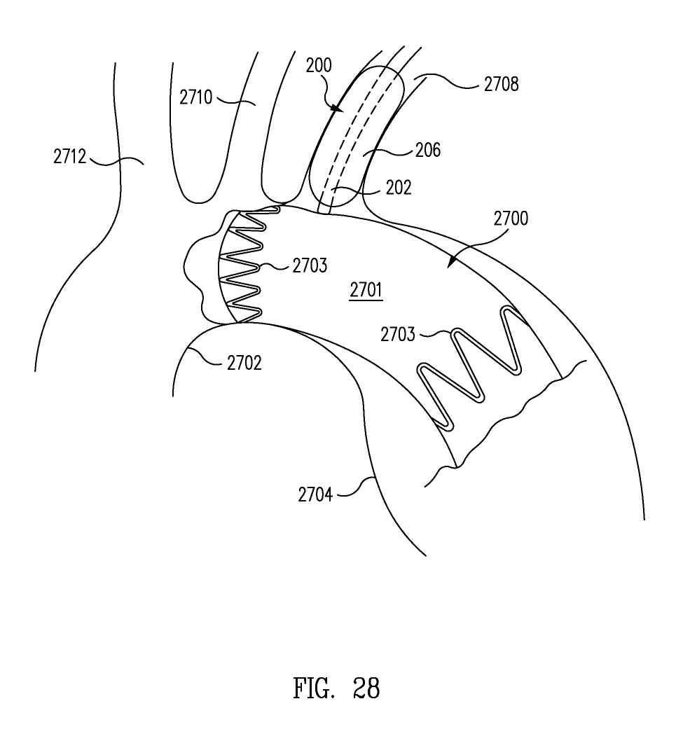

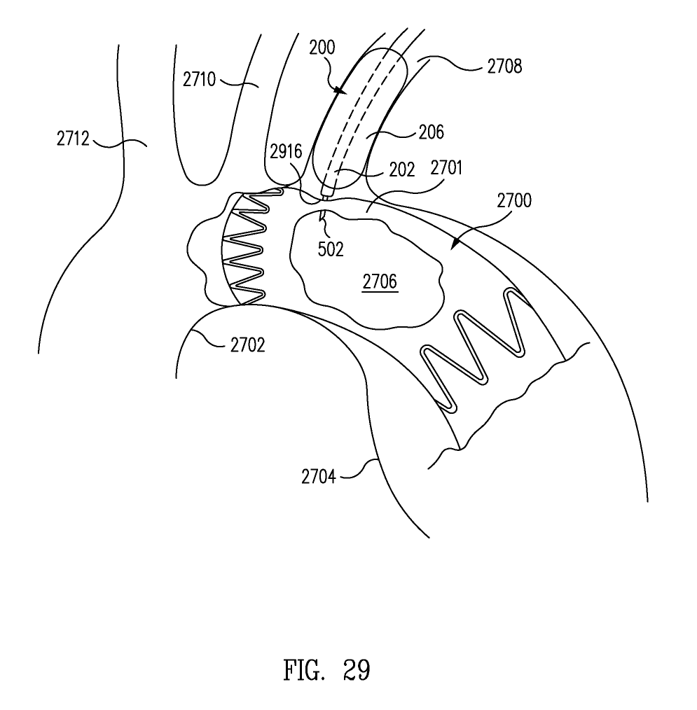

FIGS. 27, 28, 29, 30, 31, 32, 33, and 34 are perspective views illustrating formation of a collateral opening in a main stent graft 2700 and deployment of a branch prosthesis within the collateral opening in accordance with one embodiment. Referring now to FIG. 27, main stent graft 2700 is deployed into a main vessel 2702 using any one of a number of well known techniques, the particular delivery/deployment technique used is not essential. Main stent graft 2700 includes a graft material 2701, sometimes called a graft cloth, and supporting structures 2703, e.g., self-expanding stents.

Main vessel 2702, e.g., the aorta, includes an aneurysm 2704. Generally, main stent graft 2700 seals against main vessel 2702 above and below, e.g., proximally and distally to, aneurysm 2704. Accordingly, fluid flows through lumen 2706 of main stent graft 2700 thus bypassing and excluding aneurysm 2704.

Branching off main vessel 2702 are three branch vessels 2708, 2710, 2712, e.g., the subclavian, the common carotid, and the brachiocephalic trunk. In the example illustrated in FIG. 27, main stent graft 2700 is deployed proximally to branch vessel 2708 covering (over) the ostium of branch vessel 2708 restricting blood flow to branch vessel 2708. However, in another example, a main stent graft is deployed proximally to all three of branch vessels 2708, 2710, 2712. For example, the proximal end of the main stent graft is illustrated by the dashed line 2714 in FIG. 27 and the procedure described in reference to FIGS. 27 to 34 is repeated to provide collateral flow to each of branch vessels 2708, 2710, 2712.

Referring now to FIGS. 26 and 27 together, anchoring balloon 206 is advanced through branch vessel 2708 to be adjacent to main stent graft 2700. Marker band 210 facilitates positioning of anchoring balloon 206. As illustrated in FIG. 27, anchoring balloon 206 typically tracks the outer diameter of curvature of branch vessel 2708, i.e., is not centered within branch vessel 2708.

In accordance with this example, fully assembled user interface 2200 as illustrated in FIG. 26 with needle dilator assembly 2301 fastened to anchor balloon catheter 200 is manipulated to advance anchoring balloon 206 to main stent graft 2700. As discussed above, needle actuator handle 2300 and dilator actuator handle 2500 are locked to actuator shaft 2302 in their retracted position thus preventing the needle and dilator from inadvertently being deployed during advancement of anchoring balloon 206. Further, as discussed above, slots 510, 810 within slotted hypotubes 504, 804 facilitate bending of slotted hypotubes 504, 804 during advancement to main stent graft 2700.

Referring now to FIGS. 26, 27 and 28 together, anchoring balloon 206 is inflated, e.g., by injecting a saline solution into balloon inflation port 2520. Inflation of anchoring balloon 206 centers inner member 202 approximately parallel with the axis of branch vessel 2708. At the same time, anchoring balloon 206 exerts an outward radial force over a large contact area of branch vessel 2708 thus anchoring anchoring balloon 206 within branch vessel 2708. The centering and anchoring of anchoring balloon 206 is achieved due to the rectangular and compliant design of anchoring balloon 206 as discussed above. This insures that both needle 502 and dilator 802 puncture/dilate graft material 2701 of main stent graft 2700 centered to and substantially parallel with the axis of branch vessel 2708 as discussed further below.

Referring now to FIGS. 5, 26 and 29 together, needle 502 is advanced to protrude from inner member 202 and to pierce graft material 2701 of main stent graft 2700. This forms a needle hole 2916, sometimes called an opening or aperture, within graft material 2701 of main stent graft 2700. Needle hole 2916 is centered with respect to the axis of branch vessel 2708 thus minimizing propagation of a rent from the final collateral opening formed from needle hole 2916 and/or damage to the branch prosthesis deployed therein.

In one example, to advance needle 502, actuation button 2318 of needle actuator handle 2300 is depressed, e.g., by the physician to unlock needle actuator handle 2300 from actuator shaft 2302. Needle actuator handle 2300 is distally slid on actuator shaft 2302 translating to distal longitudinal motion (advancement) of slotted hypotube 504 and needle 502. Slotted hypotube 504 has strength in compression to force needle 502 through graft material 2701 of main stent graft 2700. Needle actuator handle 2300 contacts distal stop 2316 thus preventing further distal motion of needle actuator handle 2300 and needle 502. In this manner, inadvertent puncture of the opposite side of graft material 2701 and main vessel 2702 is prevented. Actuation button 2318 is then released thus locking needle actuator handle 2300 in its extended position to actuator shaft 2302.

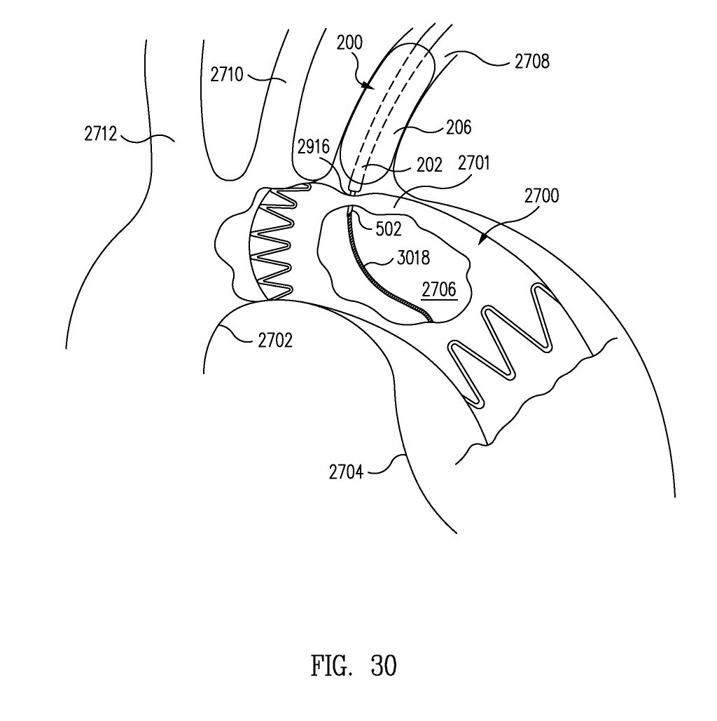

Referring now to FIGS. 5, 26 and 30 together, a guide wire 3018 is passed through needle 502 and into lumen 2706 of main stent graft 2700. More particularly, guide wire 3018 is passed into slotted hypotube 504 through its proximal end, passed through slotted hypotube 504, needle 502 and into lumen 2706 of main stent graft 2700 as illustrated in FIG. 30.

Referring now to FIGS. 5, 26, 30 and 31 together, needle 502 is retracted back into inner member 202. In one example, to retract needle 502, actuation button 2318 of needle actuator handle 2300 is depressed, e.g., by the physician to unlock needle actuator handle 2300 from actuator shaft 2302. Needle actuator handle 2300 is proximally slid on actuator shaft 2302 translating to proximal longitudinal motion (retraction) of slotted hypotube 504 and needle 502. Slotted hypotube 504 has strength in tension to pull needle 502 from graft material 2701 of main stent graft 2700. Needle actuator handle 2300 contacts proximal stop 2314 thus preventing further proximal motion of needle actuator handle 2300 and needle 502. Actuation button 2318 is then released thus locking needle actuator handle 2300 in its retracted position to actuator shaft 2302.

Although retraction of needle 502 prior to advancement of dilator 802 is set forth below, in another example, needle 502 is left in its extended position through graft material 2701 and dilator 802 is advanced over needle 502 to dilate needle hole 2916.

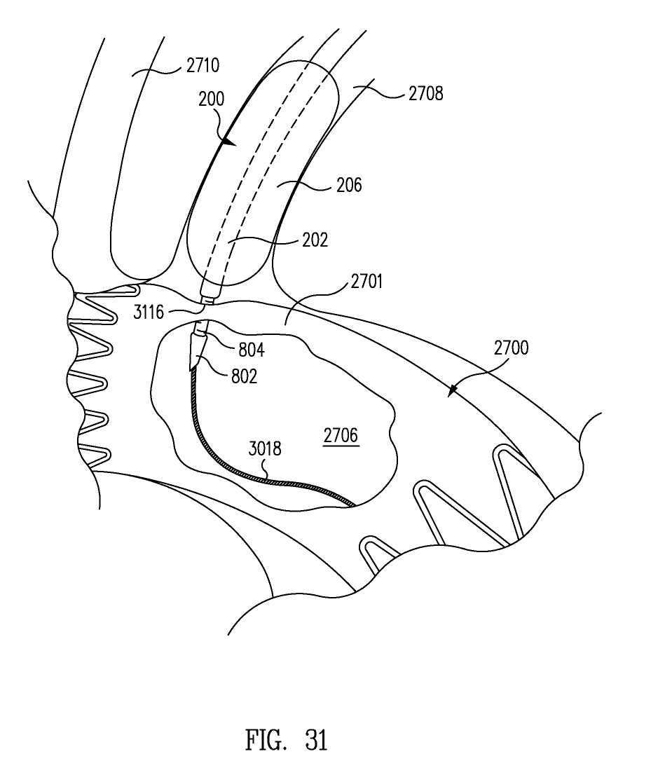

In accordance with the example where needle 502 is retracted, dilator 802 is advanced through needle hole 2916 thus dilating (enlarging, sometimes called increasing in diameter) needle hole 2916 (FIG. 30) to form a dilated needle hole 3116 (FIG. 31). Dilated needle hole 3116 has a larger diameter than needle hole 2916.

Further, distal tapering exterior surface 814 is polished thus minimizing the deployment force necessary to force dilator 802 through graft material 2701 of main graft 2700. More particularly, distal tapering exterior surface 814 gradually increases the diameter of (dilates) needle hole 2916 as graft material 2701 is forced outwards on the taper of distal tapering exterior surface 814. Further, as the outer diameter of shoulder 816 is greater than the outer diameter of slotted hypotube 804, the diameter of dilated needle hole 3116 is greater than the outer diameter of slotted hypotube 804. This minimizes friction between slotted hypotube 804 and graft material 2701 of main stent graft 2700.

In one example, to advance dilator 802, an actuation button 2318A of dilator actuator handle 2500 is depressed, e.g., by the physician to unlock dilator actuator handle 2500 from actuator shaft 2302. Dilator actuator handle 2500 is distally slid on actuator shaft 2302 translating to distal longitudinal motion (advancement) of slotted hypotube 804 and dilator 802. Slotted hypotube 804 has strength in compression to force dilator 802 through graft material 2701 of main stent graft 2700. In one example, slotted hypotube 804 can bear loads of at least 2 lbf. Dilator actuator handle 2500 contacts engagement cap 2516 thus preventing further distal motion of dilator actuator handle 2500 and dilator 802. Further, as dilator 802 is advanced over guide wire 3018, dilator 802 tracks guide wire 3018 thus preventing inadvertent puncture of the opposite side of graft material 2701 and main vessel 2702 by dilator 802. Actuation button 2318A is then released thus locking dilator actuator handle 2500 in its extended position to actuator shaft 2302, if desired.

After dilation of needle hole 2916, dilator 802 is retracted out of main stent graft 2700 and back within inner member 202. In one example, to retracted dilator 802, actuation button 2318A of dilator actuator handle 2500 is depressed, e.g., by the physician, to unlock dilator actuator handle 2500 from actuator shaft 2302. Dilator actuator handle 2500 is proximally slid on actuator shaft 2302 translating to proximal longitudinal motion (retraction) of slotted hypotube 804 and dilator 802. Slotted hypotube 804 has strength in tension to pull dilator 802 from graft material 2701 of main stent graft 2700. Dilator actuator handle 2500 contacts distal stop 2316 (which forms the proximal stop for dilator actuator handle 2500 as discussed above) thus preventing further proximal motion of dilator actuator handle 2500 and dilator 802. Actuation button 2318A is then released thus locking dilator actuator handle 2500 to actuator shaft 2302.

Proximal tapered exterior surface 818 facilitates retraction of dilator 802 through graft material 2701 of main stent graft 2700 without disturbance of (catching on) graft material 2701. This minimizes the retraction force necessary to retract dilator 802.

Referring now to FIGS. 26, 31 and 32 together, locking snap 2526 is removed thus releasing needle dilator assembly 2301 from anchor balloon catheter handle 2518. Needle dilator assembly 2301 including the needle and dilator are removed from anchor balloon catheter 200.

Although a separate needle 502 and dilator 802 are set forth above for forming dilated needle hole 3116, in another example, a similar dilated needle hole is formed using hybrid needle dilators 1402, 1702 of FIGS. 14, 17, respectively. In accordance with these examples, hybrid needle dilators 1402, 1702 puncture the graft material of the main stent graft to form an opening and then dilate the opening during a single advancement of hybrid needle dilators 1402, 1702.

Anchoring balloon 206 is deflated, slightly retracted, and re-inflated. A dilation balloon 3220 is passed through anchoring balloon catheter 200 and inserted into dilated needle hole 3116. Dilated needle hole 3116 has a diameter sufficient to accommodate dilation balloon 3220. Dilation balloon 3220 is inflated thus further enlarging dilated needle hole 3116 forming a collateral opening 3222 within graft material 2701 of main stent graft 2700.

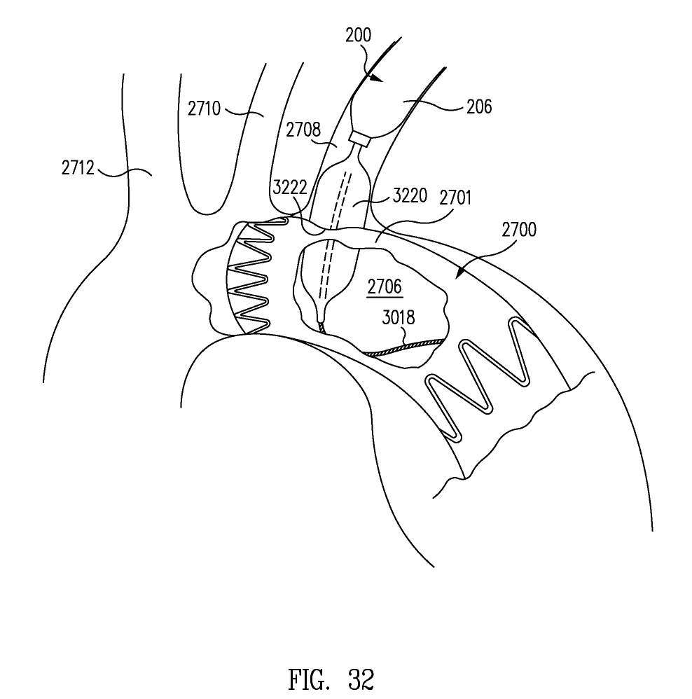

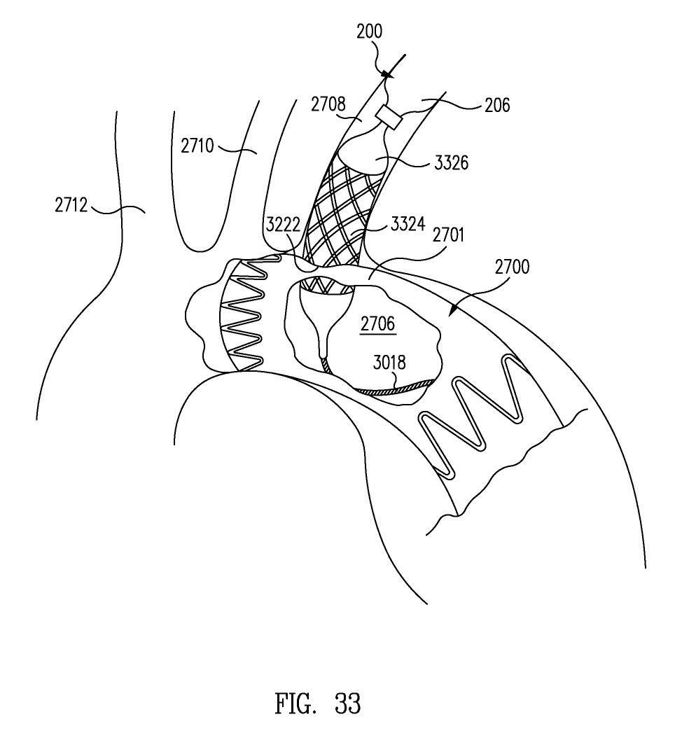

Referring now to FIGS. 32 and 33 together, dilation balloon 3220 is removed from anchoring balloon catheter 200. A branch prosthesis 3324, e.g., a coated or covered stent, is passed through anchoring balloon catheter 200 and inserted into collateral opening 3222. Collateral opening 3222 has a diameter sufficient to accommodate branch prosthesis 3324. Branch prosthesis 3324 is expanded within collateral opening 3222, e.g., using a balloon 3326, although a self-expanding prosthesis is used in other examples.

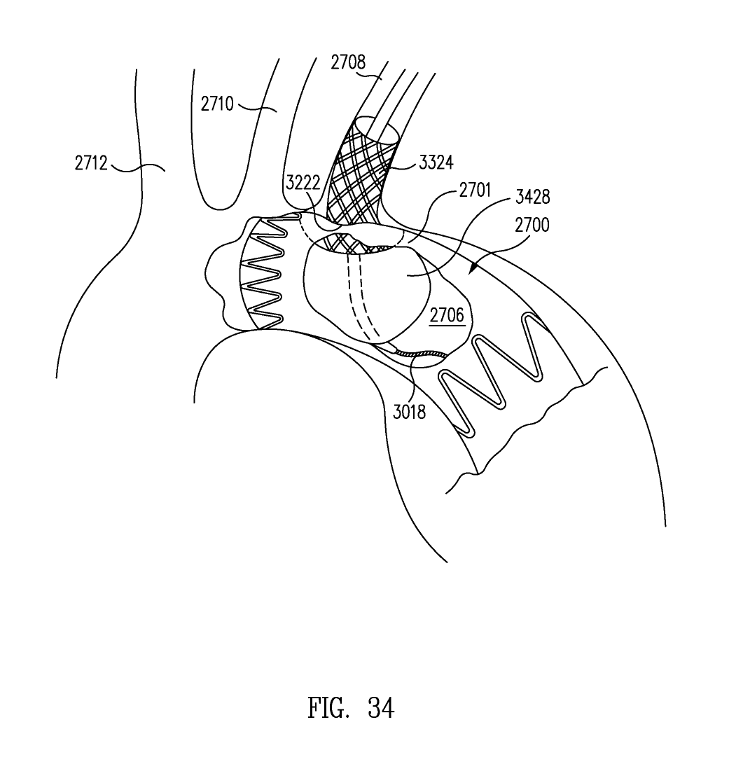

Referring now to FIGS. 33 and 34 together, balloon 3326 is removed from anchoring balloon catheter 200. Branch prosthesis 3324 remains and extends from collateral opening 3222 and into branch vessel 2708. A branch prosthesis flaring balloon 3428, e.g., a spherical occlusion balloon, is passed through anchoring balloon catheter 200 and inserted into branch prosthesis 3324 inside of lumen 2706 of main stent graft 2700. Branch prosthesis flaring balloon 3428 is inflated flaring the end of branch prosthesis 3324 thus locking branch prosthesis 3324 to main stent graft 2700.

Branch prosthesis flaring balloon 3428 is deflated and removed. Anchoring balloon 206 is deflated and removed. In one example, anchoring balloon 206 is removed earlier in the procedure, e.g., after retraction of dilator 802 (after the procedure illustrated in FIG. 31).

The drawings and the forgoing description give examples according to the present invention. The scope, however, is by no means limited by these specific examples. Numerous variations, whether explicitly given in the specification or not, such as differences in structure, dimension, and use of material, are possible.

What is claimed is:

1. A method comprising:
deploying a main stent graft within a main vessel over a branch vessel branching from said main vessel, said main stent graft comprising a graft material;
advancing an anchoring balloon of an anchoring balloon catheter through said branch vessel to be adjacent to said main stent graft, said anchoring balloon catheter further comprising an inner member defining an inner member lumen therein;
inflating said anchoring balloon to center said inner member parallel with an axis of said branch vessel and to anchor said anchoring balloon within said branch vessel;
advancing a needle assembly located within a dilator assembly located within said inner member lumen to pierce said graft material of said main stent graft with a needle of said needle assembly forming a needle hole in said graft material; and
advancing said dilator assembly to dilate said needle hole with a dilator of said dilator assembly.

2. The method of claim 1 wherein said anchoring balloon is a compliant balloon.

3. The method of claim 1 wherein said anchoring balloon comprises a cylindrical outer surface.

4. The method of claim 1 wherein said needle assembly further comprises a slotted hypotube attached to said needle.

5. The method of claim 4 wherein said slotted hypotube comprises a plurality of alternated laser C-slots.

6. The method of claim 1 wherein said dilator assembly further comprises a slotted hypotube attached to said dilator.

7. The method of claim 6 wherein said slotted hypotube comprises a plurality of alternated laser C-slots.

8. The method of claim 1 further comprising retracting said needle prior to said advancing said dilator assembly.

9. The method of claim 1 wherein said dilator comprises a bevel that provides a smooth transition of said dilator into said needle hole.

10. The method of claim 1 wherein said dilator comprises a distal tapering exterior surface that dilates said needle hole.

11. The method of claim 10 further comprising retracting said dilator out of said main stent graft, said dilator comprising a proximal tapering exterior surface that provides a gradual diameter transition to prevent said graft material from catching on said dilator.

12. The method of claim 1 wherein said advancing a needle assembly comprises advancing a needle actuator handle coupled to said needle assembly.

13. The method of claim 1 wherein said advancing a dilator assembly comprises advancing a dilator actuator handle coupled to said dilator assembly.

14. A dilator assembly comprising a dilator for dilating a needle hole in a graft material of a main stent graft, said dilator comprising:
a bevel;
a distal tapering exterior surface;
a shoulder, said distal tapering exterior surface extending from said bevel to said shoulder; and
a proximal tapering exterior surface extending from said shoulder.

15. The dilator assembly of claim 14 wherein said dilator comprises metal.

16. The dilator assembly of claim 14 wherein said bevel defines a distal surface of said dilator.

17. The dilator assembly of claim 14 wherein said bevel is an elliptical annular surface having a varied thickness.

18. The dilator assembly of claim 17 wherein a distance between an outer periphery of said bevel and an inner periphery of said bevel is minimum at a distal end of said bevel and maximum at a proximal end of said bevel and gradually increases between said distal end and said proximal end.

19. The dilator assembly of claim 14 wherein said distal tapering exterior surface increasing tapers proximally from said bevel to said shoulder.

20. The dilator assembly of claim 14 wherein said shoulder is a maximum diameter portion of said dilator.

21. The dilator assembly of claim 14 further comprising a slotted hypotube coupled to said dilator, said shoulder having a greater outer diameter than an outer diameter of said slotted hypotube.

22. The dilator assembly of claim 14 wherein said dilator further comprises a sharp tip at a distal end of said dilator.

23. A method comprising:
deploying a main stent graft within a main vessel over a branch vessel branching from said main vessel, said main stent graft comprising a graft material;
advancing an anchoring balloon of an anchoring balloon catheter through said branch vessel to be adjacent to said main stent graft, said anchoring balloon catheter further comprising an inner member defining an inner member lumen therein;
inflating said anchoring balloon to center said inner member parallel with an axis of said branch vessel and to anchor said anchoring balloon within said branch vessel; and
advancing a hybrid needle-dilator assembly located within said inner member lumen to pierce said graft material of said main stent graft with a sharp tip of a hybrid needle-dilator of said hybrid needle-dilator assembly forming a needle hole in said graft material and to dilate said needle hole with said hybrid needle-dilator.

24. A needle dilator guiding catheter assembly comprising:
an anchoring balloon catheter comprising:
an inner member defining an inner member lumen; and
an anchoring balloon mounted on said inner member, said inner member having a strength sufficient to prevent collapse of said inner member and constriction of said inner member lumen when said anchoring balloon is inflated;
a dilator assembly configured to fit in said inner member lumen, said dilator assembly defining a dilator assembly lumen therein, said dilator assembly comprising:
a slotted hypotube; and
a dilator mounted to said slotted hypotube;
a needle assembly configured to fit within said dilator assembly lumen, said needle assembly defining a guidewire lumen therein, said needle assembly comprising:
a slotted hypotube; and
a needle mounted to said slotted hypotube of said needle assembly; and
a user interface comprising:
an anchoring balloon catheter handle of said anchoring balloon catheter;
a needle actuator handle coupled to said needle assembly;
a dilator actuator handle coupled to said dilator assembly; and
an actuator shaft comprising:
a proximal stop for limiting a proximal travel of said needle actuator handle;
a distal stop for limiting a distal travel of said needle actuator handle and for limiting a proximal travel of said dilator actuator handle; and
an engagement cap for limiting a distal travel of said dilator actuator handle.

25. The needle dilator guiding catheter assembly of claim 24 wherein said actuator shaft further comprises:
a first retracted position locking engagement groove for locking said needle actuator handle in a retracted position;
a first extended position locking engagement groove for locking said needle actuator handle in an extended position;
a second retracted position locking engagement groove for locking said dilator actuator handle in a retracted position; and
a second extended position locking engagement groove for locking said dilator actuator handle in an extended position.
